(19) United States
(12) Patent Application Publication
(10) Pub. No.: US 2021/0290560 A1
(43) Pub. Date: Sep. 23, 2021

(54) TRANSDERMAL THERAPEUTIC SYSTEM FOR DISPENSING SCOPOLAMINE WITHOUT A MEMBRANE

(71) Applicant: LTS LOHMANN THERAPIE-SYSTEME AG, Andernach (DE)

(72) Inventors: Sandra WIEDERSBERG, Steigra (DE); Gerd HOFFMANN, Neuwied (DE); Walter MÜLLER, Andernach (DE)

(21) Appl. No.: 17/264,265

(22) PCT Filed: Jul. 31, 2019
(86) PCT No.: PCT/EP2019/070667

§ 371 Date: Jan. 28, 2021

(30) Foreign Application Priority Data
Jul. 31, 2018 (DE) 10 2018 118 507.3

(51) Int. Cl. A61K 9/70; A61K 31/46

(52) U.S. Cl. CPC A61K 9/7069; A61K 9/7092; A61K 31/46

(57) ABSTRACT

The present invention relates to a transdermal therapeutic system without a release-determining membrane for delivering scopolamine, comprising a skin contact layer, an active substance-containing reservoir layer, an active substance-impermeable backing layer, and optionally a protective layer which is detachable from the skin contact layer, wherein the skin contact layer is in direct contact with the active substance-containing reservoir layer. The skin layer or the skin layer and the reservoir layer may be equipped with adhesive strength-increasing additives, for example silicone oils. Furthermore, the system may be equipped with an active substance-free over-patch in order to ensure a sufficient adhesion onto the skin over the application duration. In comparison to comparable systems with a release-determining membrane, a similar bioavailability of the active substance was achieved, such that these systems are bioequivalent to the membrane-containing systems. The present invention additionally relates to the aforementioned transdermal therapeutic systems for use in the treatment of motion sickness and/or post-operative nausea as well as to methods for producing same.

The present invention relates to a transdermal therapeutic system without a release-determining membrane for delivering scopolamine, based on a multi-layer system comprising a skin contact layer, an active substance-containing reservoir layer, an active substance-impermeable backing layer, and optionally a protective layer which is detachable from the skin contact layer. The present invention also relates to a method for producing such transdermal therapeutic systems as well as to corresponding systems for use in the treatment of motion sickness and/or post-operative nausea.

### PRIOR ART

Scopolamine is a known active substance which, with the aid of a patch, is suitable for transdermal application with systemic effect. This is what is known as an antiemetic, which is preferably used to prevent nausea and vomiting, for example as a result of repeated passive changes to equilibrium during travel. The therapeutic advantage of a transdermal administration lies in the fact that the active substance is supplied slowly and continuously under control by the transdermal system, thus making it possible to reliably provide the relatively narrow therapeutic window for scopolamine and to set a therapeutically effective plasma level, without the side effects that occur in the event of an overdose, such as dryness of the mouth, nausea and sensitivity of the eyes to light.

Transdermal administration forms of scopolamine are available on the market, for example under the trade name Transderm Scop® in the USA. This patch, which is described in U.S. Pat. No. 4,904,475 A, consists of four layers: a carrier film, a drug reservoir, a membrane controlling the rate of active substance release, and a contact adhesive loaded with active substance. In addition, a membrane patch is available in Europe under the trade name Scopoderm TTS®, which has a membrane based on microporous polypropylene, which controls the rate of release. A further transdermal scopolamine patch product was recently presented by the company Aveva/Perrigo. This patch consists of a backing layer, a reservoir layer applied thereto, an ethylene-vinyl acetate copolymer membrane, an adhesive skin contact layer, and a detachable protective layer.

The relatively complicated structure of these release systems leads to increased production costs, since the production process is relatively complex. On the whole, a relatively high time expenditure is also necessary for the production of corresponding application systems. A further disadvantage of the product known as Transderm Scop® TTS is the cold flow, which causes clothing or the hands to be dirtied with the “toxic” substance when the TTS is applied and/or worn. In addition, in order to be able to provide the active substance in sufficient quantity already at the start of the application period, the system must be thermodynamically oversaturated, which may lead, during storage, to a disadvantageous formation of crystals.

The systems available on the market are also loaded over their entire surface with active substance, and therefore application of the transdermal therapeutic system to the skin may lead to a cross contamination (contact between hands and active substance and subsequent contact between hands and other objects/areas of the body). Further contact of the hands, for example with the eyes, may lead to an undesirable dilation of the pupils on account of the active substance properties.

EP 1 011 674 A1 describes a transdermal therapeutic system for administering scopolamine which, similarly to the Transderm Scop® product, contains a membrane which controls the delivery rate of the active substance and which consists of an ethylene-vinyl acetate copolymer. In this system, an amine-resistant silicone polymer is used as matrix for the active substance.

German patent application DE 198 25 499 A1 discloses active substance-containing patches which contain one or more active substances, for example scopolamine, in the form of spun fibres or yarns in an adhesive applied to a carrier material. DE 696 22 780 T2 describes special active substance-containing patches in which an active substance such as scopolamine is enclosed in microcapsules. Both of the above-mentioned transdermal therapeutic systems may contain various additives in addition to the active substance.

DE 10 2004 059 674 B4 describes a transdermal system for delivering scopolamine, from which 500 μm to 2 mg of scopolamine are to be delivered over a period of time of at least up to 72 h, although this system does not contain the membrane controlling the rate of release. The matrix polymer forming the basis of the disclosure of DE 10 2004 059 674 64 is based on styrene-butadiene copolymer adhesives, especially on a styrene-butadiene-styrene adhesive. For the latter system, however, a significantly increased release of scopolamine from the TTS was observed in comparison to the Transderm Scop® reference system.

Compared to the products currently on the market, there is a need for a transdermal application form for scopolamine which has a relatively simple structure and with which it is possible to dispense with a membrane controlling the release of scopolamine. In addition, there is a need for administration system for scopolamine which, in the initial phase following the fixing of the delivery system to the skin, provides a high initial flow, so that a therapeutically effective level of active substance may be built up relatively quickly in the body. On the other hand, the transdermal application system should ensure a constant supply of active substance and a constant effective level of active substance over the wearing period (for example three days).

There is also a need for an administration system with which the risk of cross contamination with scopolamine is reduced. The present invention addresses these needs.

### DETAILED DESCRIPTION

According to a first aspect, the present invention relates to a transdermal therapeutic system without a release-determining membrane for delivering scopolamine, comprising a skin contact layer, and active substance-containing reservoir layer, an active substance-impermeable backing layer, and optionally a protective layer which is detachable from the skin contact layer, wherein the skin contact layer is in direct contact with the active substance-containing reservoir layer.

As active substance, the transdermal therapeutic system contains scopolamine as free base, although small proportions (i.e. 10 wt. % or less) of scopolamine salts, such as the hydrohalide and especially hydrobromide, are not harmful. In the context of the present invention, however, the use exclusively of the free base of scopolamine as active substance is most preferred.

In order to achieve a high initial flow during the time following application of the TTS, the skin contact layer expediently has a high thermodynamic activity. This is achieved expediently in that the skin contact layer contains scopolamine as dissolved active substance, the thermodynamic activity of the active substance being kept high as a result of the fact that the permeation enhancer/solubilising agent diffuses into the skin more quickly than the active substance. The active substance diffusing more slowly than the permeation enhancer/solubilising agent remains in solution and has a high thermodynamic activity as a result of the reduced content of permeation enhancer/solubilising agent, so that a high initial flow is generated within a short space of time (i.e. approximately 16 hours after application). An active substance content in the range of from 1.2 to 13 wt. % and preferably 9 to 11 wt. % may be stated as a suitable active substance content for the skin contact layer. A range of from 0.06 to 0.5 mg and preferably 0.1 to 0.4 mg may be stated as a suitable quantity of active substance in the skin contact layer.

The majority of the active substance scopolamine in the transdermal therapeutic system according to the invention is located in the reservoir layer. Consequently, this contains a significantly greater quantity of active substance in comparison to the skin contact layer, and an active substance quantity in the range of from 1 to 1.44 mg and preferably 1.04 to 1.3 mg may be stated as being especially favourable. The active substance content in the reservoir layer is expediently 7 to 20 wt. % and preferably 8 to 11 wt. %.

The active substance in the active substance-containing reservoir layer may be either fully dissolved or also partially suspended. If the active substance is partially suspended, it is characterised expediently by a favourable particle size, which is defined by a D10 of approximately 5 μm, a D90 of approximately 15 μm and a D99 of approximately 35 μm. The term “approximately” in this context refers especially to a deviation from the stated value by ±50%, preferably by ±30%, more preferably by ±20% and even more preferably by ±10%. As a result of this particle size, the continuous delivery of a therapeutically effective quantity of active substance over the entire period of use of the transdermal therapeutic system is promoted, without the need for a release-determining membrane for this purpose. In the context of this application, D10, D90 and D99 are determined with the aid of laser diffraction, as described in European Pharmacopoeia 8.0 (2013), Chapter 2.9.31. The designation D (numerical value) stands for the numerical value for the proportion of particles (in percent) that are smaller than or the same size as the stated particle size (i.e. with a D90 of 15 μm, 90% of the particles have a size of ≤15 μm).

So that the transdermal therapeutic system may provide a therapeutically effective quantity of scopolamine over a period of time of 72 h, it is also expedient if it contains a total quantity of approximately 0.8 to 2.0 mg, preferably 1.2 to 1.5 mg scopolamine, and especially preferably 1.35 to 1.45 mg scopolamine.

A polymer that has a high diffusibility, for example silicone adhesive polymers, is suitable as matrix polymer for the scopolamine in the skin contact layer and in the reservoir layer.

The skin contact layer and the reservoir layer are preferably based on adhesion-promoting polymers, which may be the same or different for the skin contact layer and the reservoir layer. For reasons of compatibility, however, it is preferred within the scope of the present invention if the skin contact layer and the reservoir layer are based on the same kind of adhesion-promoting polymers, it being especially preferred if the skin contact layer and the reservoir layer are based on the same polymer, i.e. in this case the adhesive polymers of the skin contact layer and of the reservoir layer are identical.

The expression “based on” in the present context means, especially, that the majority of the layer in question is attributable to the stated adhesion-promoting polymer, i.e. that this accounts for the greatest mass fraction in the composition of the layer. The mass fraction may possibly also be 40 wt. % or less, if all other components of the composition are present in quantities that are much lower than 40 wt. %. However, it is preferred if the adhesion-promoting polymer accounts for a proportion of at least 50 wt. %, more preferably at least 60 wt. %, and even more preferably at least 70 wt. %. It is very especially preferred if the layer in question which is based on adhesion-promoting polymer as base polymer contains this polymer as adhesion-promoting polymer substantially (i.e. to an extent of at least 90 wt. % and especially to an extent of at least 99 wt. %) and especially exclusively.

A polymer that is especially suitable within the scope of the present invention for use in the skin contact layer and the reservoir layer is an amine-resistant silicone adhesive polymer. Amine-resistant silicone adhesive polymers are characterised in that they do not have any free SiOH groups. Corresponding silicone adhesive polymers are obtainable from regular SiOH group-containing silicone adhesive polymers by conversion of the SiOH groups into SiOCH₃ groups. Silicone adhesive polymers of this kind are described, for example, in EP-A-0 180 377.

The stated silicone adhesive polymers additionally have the advantage that they are highly soluble in non-polar solvents, such as n-heptane, whilst these solvents have only a low dissolving capacity for scopolamine base. With use of silicone adhesive polymers of the aforementioned kind as base polymer, it is therefore possible to incorporate the active substance into a solution of the silicone adhesive polymers without the active substance being completely dissolved in the adhesive polymer solution. In addition, the solvent is removed at temperatures lying below the melting point of scopolamine base.

In addition to scopolamine, the reservoir layer and/or the skin contact layer may contain one or more permeation enhancers. Permeation enhancers reduce the barrier effect of the human skin and thus increase the permeation of the active substance into the skin.

Fatty acids, alcohols or ethers are preferably used as permeation enhancers. Oleic acid, 2-(2-ethoxyethoxy-ethanol (Transcutol) and dipropylene glycol have proven to be especially suitable in this regard and, in the used concentrations, do not lead to skin irritations and are compatible with silicone adhesive polymers. An additional advantage of these substances is that they increase the very low solubility of scopolamine base in the silicone adhesive polymers.

If permeation enhancers are to be provided, suitable proportions may be within a range of from 3 to 12 wt. %, and especially 6 to 10 wt. %.

Furthermore, the reservoir layer and/or the skin contact layer may contain solubility enhancers, which increase the solubility for scopolamine in the reservoir layer and/or in the skin contact layer. A relatively high quantity of dissolved active substance over a relatively small area (for example approximately 1 cm²) may be possible as a result of the addition of solubility enhancers.

Suitable solubility enhancers are surfactants, for example. By adding small quantities of a surfactant, it may thus be ensured that a therapeutically effective level of active substance is established in the blood of the person wearing the system already after a short period of time. A surfactant that is especially suitable within the scope of the present invention is, for example, polyoxyethylene (4) lauryl ether, which is commercially available, for example as BrijL4.

With regard to the aforementioned permeation enhancers and solubility enhancers, it should be noted that compounds may be effective both as permeation enhancers and solubility enhancers. In this case, the reservoir layer and/or the skin contact layer contains permeation enhancers and solubility enhancers in the form of a compound.

If provided, the solubility enhancer(s) are expediently incorporated in the reservoir layer and/or the skin contact layer with a content in the range of from 0.5 to 5 wt. % and preferably 1 to 3 wt. %.

In order to distribute permeation enhancers and/or solubility enhances uniformly in the adhesive matrix, these may be thickened using a thickening agent. Suitable thickening agents are, for example, cellulose derivatives such as ethyl cellulose. The stability of the transdermal therapeutic system may be influenced advantageously by the addition of thickening agents. If provided, the one or more thickening agents are expediently incorporated into the reservoir layer and/or the skin contact layer with a content in the range of from 0.1 to 2 wt. %, and preferably 0.5 to 1.2 wt. %.

Other additives, such as silicone oils, may be used in order to improve the physical properties of the adhesive layer, for example its tack. This may be expedient especially for the skin contact layer, in order to improve the adhesion of the transdermal therapeutic system to the skin. In a preferred embodiment, the skin contact layer of the transdermal therapeutic system described here therefore contains a tack enhancer, preferably in the form of a silicone oil.

If provided, the tack enhancer(s) are incorporated in the reservoir layer and/or the skin contact layer expediently with a content in the range of from 3 to 15 wt. %, and preferably 8 to 12 wt. %.

As is clear from the above, there are overlaps between the components of the skin contact layer and the reservoir layer. The compositions of the skin contact layer and the reservoir layer, however, generally are not identical, i.e. there are differences in the components of the composition or in their proportion in the composition, for example a higher scopolamine proportion in the reservoir layer.

The transdermal therapeutic system according to the present invention is preferably designed such that, after application to the human skin, approximately 1 mg of active substance is released from the transdermal therapeutic system in 72 h.

Alternatively or additionally, the transdermal therapeutic system according to the present invention is designed so that the active substance is released from the transdermal therapeutic system over a period of time from 20 h to 72 h at a release rate of from 1.5 to 15 μg/cm²/h and preferably 3 to 10 μg/cm²/h. The area-based designation “cm²” in this case denotes the surface over which the skin contact layer is in contact with the skin.

It is very especially preferred if the transdermal therapeutic system according to the invention is “bioequivalent” to the product Transderm Scop®, which has already been approved, since in this case it would not be necessary to undergo a separate, complex approval procedure. According to WHO, two pharmaceutical products are termed as being “bioequivalent” if they are pharmaceutically equivalent or pharmaceutical alternatives and their bioavailabilities, expressed in rate (C max and t max) and the extent of the absorption (area under the curve) after administration of the same molar dose under the same conditions are similar to such an extent that it may be assumed that their effects are substantially the same (see WHO Guidance for organizations performing in vivo bioequivalence studies. WHO Technical Report Series No. 996, 2016, Annex 9). Here, C max denotes the maximum plasma concentration and t max denotes the time until this is achieved. A drug is considered within the scope of the present invention to be “bioequivalent” to Transderm Scop® if it provides a bioavailability of from 80 to 125% of the reference drug within a 90% confidence interval.

A very especially preferred transdermal therapeutic system according to the invention, within a period of time from the application to the skin up to a time 72 h after the application, delivers approximately 1 mg of scopolamine (i.e. ±10%) at an approximately constant rate (i.e. variation of the rate of at most ±20% and preferably at most ±10%).

The contact area of the skin contact layer of the transdermal therapeutic system is dependent on the specific active substance release rate and the effective dose of the active substance, but usually lies in the range of from 0.5 to 3 cm², especially 0.8 to 2 cm², more preferably 1 to 2 cm², and even more preferably 1 to 1.8 cm².

The shape of the contact area is not subject to any relevant limitations, and therefore angular, especially such as square or rectangular shapes, but also round shapes, such as circular or oval shapes, may be provided. Within the scope of the present invention, round embodiments and especially circular embodiments are preferred.

In order to promote an adhesion of the transdermal therapeutic system over the entire period of 72 h for which it is worn and in order to prevent a cross contamination (for example of the eyes, which even with small contact quantities of scopolamine leads to pupil dilation and temporary visual impairment), the system may be equipped with an active substance-free over-patch. In comparison to the contact area of the skin contact layer on the skin, the over-patch has a larger surface, such that the over-patch protrudes beyond the entire circumference of the skin contact layer. If a transdermal therapeutic system equipped in this way is fixed to the skin, an area in which the over-patch adheres directly to the skin is thus created over the entire circumference of the skin contact layer.

The transdermal therapeutic system according to the invention is suitable especially for use in the treatment of motion sickness and/or post-operative nausea.

A further aspect of the present invention lastly relates to a method for producing a scopolamine-containing patch, as described above, comprising the steps of:
a) applying, to an active substance-impermeable backing layer, a scopolamine-containing reservoir layer comprising scopolamine, an adhesive polymer and optionally permeation enhancers, wherein the scopolamine is either completely dissolved in the reservoir layer or is present in part in the form of non-dissolved particles,
b) applying, to a detachable protective layer, a scopolamine-containing skin contact layer comprising scopolamine, an adhesive polymer and optionally permeation enhancers as well as adhesive force-enhancing substances, wherein the scopolamine is present in dissolved form in the skin contact layer,
c) laminating the skin contact layer from b) onto the reservoir layer from a).

The laminate produced in c) may then be modified with an over-patch, as described above, by connecting the over-patch to the active substance-impermeable backing layer of the laminate produced in c).

The invention will be illustrated in greater detail hereinafter with reference to examples, although these examples are not intended to have any effect on the interpretation of the scope of protection of the present invention.

### Example 1

### Production of the Reservoir Layer

To produce the reservoir layer, 13.78% of scopolamine base, 3% of oleic acid, and 83.22% of Bio-PSA 4301 (60% of polymer solid in heptane) were formulated as a suspension. The suspension was applied by doctor blade to a fluoropolymer-coated polyester film and was dried for 30 min at 40° C. The coating weight of the dried film was 90 g/m². The dried film was then covered with a 19 μm thick polyester film.

### Production of the Skin Contact Layer

To produce the skin contact layer, 3% of scopolamine base, 3% of oleic acid, and 94% of Bio-PSA 4301 were formulated as a solution. The solution was applied by doctor blade to a fluoropolymer-coated polyester film and was dried for 15 min at 40° C. The coating weight of the dried film was 40 g/m². The dried film was then laminated with the laminate of reservoir layer/polyester film, from which the fluoropolymer-coated film was removed beforehand.

This resulted in an active substance-containing laminate consisting of 19 μm of polyester top film, reservoir layer, skin contact layer, and fluoropolymer-coated polyester film.

Patches measuring 1 cm² in size were punched out from the overall laminate.

### Example 2

The production was performed similarly to Example 1, with the skin contact layer being formulated with a proportion of 1.2% scopolamine, 3% oleic acid, and 95.8% Bio-PSA 4301, and the reservoir layer being formulated with a proportion of 16.25% scopolamine, 3% oleic acid, and 80.75% Bio-PSA 4301. The skin contact layer was produced with a weight per unit area of 50 g/m² and the reservoir layer was produced with a weight per unit area of 80 g/m².

### Example 3

### Production of the Reservoir Layer

To produce the reservoir layer, 10.4% of scopolamine base, 8% of Transcutol, 1.1% ethyl cellulose, 2% BrijL4, and 78.5% Bio-PSA 4201 were formulated as a dispersion. The dispersion was applied by doctor blade to a fluoropolymer-coated polyester film and was dried for 11 min at room temperature. The coating weight of the dried film was 100 g/m². The dried film was then covered with a 19 μm thick polyester film.

### Production of the Skin Contact Layer

To produce the skin contact layer, 10% of scopolamine base, 7.7% of Transcutol, 1% ethyl cellulose, 2% BrijL4, 10.6% silicone oil, and 68.7% Bio-PSA 4201 were formulated as a dispersion. The dispersion was applied by doctor blade to a fluoropolymer-coated polyester film and was dried for 4 min at room temperature. The coating weight of the dried film was 40 g/m². The dried film was then laminated with the laminate of reservoir layer/polyester film, from which the fluoropolymerised film was removed beforehand.

### Example 4

### Production of the Reservoir Layer

To produce the reservoir layer, 8% of scopolamine base, 13% of dipropylene glycol, 0.7% hydroxypropyl cellulose, 2% BrijL4, and 76.2% Bio-PSA 4201 were formulated as a dispersion. The dispersion was applied by doctor blade to a fluoropolymer-coated polyester film and was dried for 14 min at room temperature. The coating weight of the dried film was 130 g/m². The dried film was then covered with a 19 μm thick polyester film.

### Production of the Skin Contact Layer

To produce the skin contact layer, 10% of scopolamine base, 16.3% of dipropylene glycol, 0.7% hydroxypropyl cellulose, 2% BrijL4, 9.5% silicone oil, and 61.6% Bio-PSA 4201 were formulated as a dispersion. The dispersion was applied by doctor blade to a fluoropolymer-coated polyester film and was dried for 4 min at room temperature. The coating weight of the dried film was 40 g/m². The dried film was then laminated with the laminate of reservoir layer/polyester film, from which the fluoropolymer-coated film was removed beforehand.

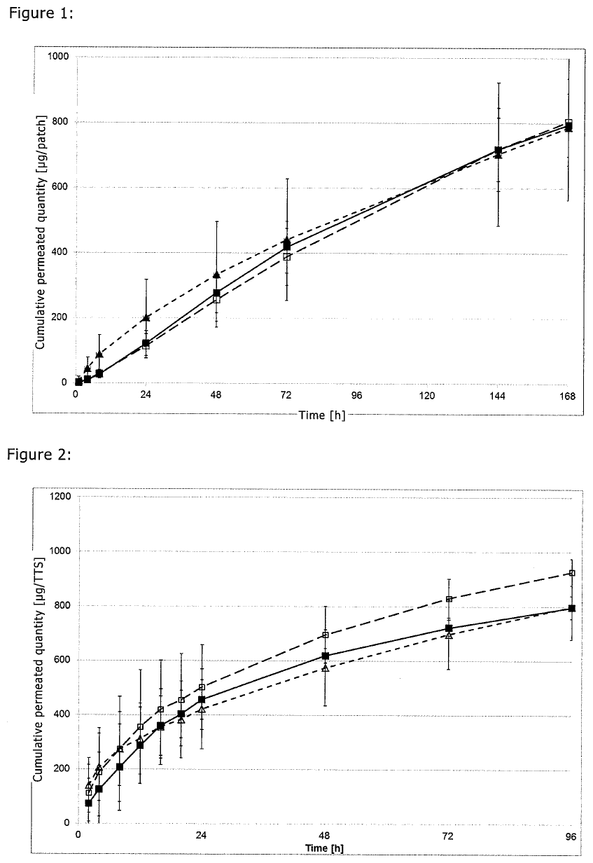

The results of comparative permeation tests between a commercially conventional comparison preparation (Transderm Scop®) and the systems according to the invention are shown in graph form in FIG. 1 and FIG. 2.

In FIG. 1 the permeation profiles, determined as cumulative quantity of permeated scopolamine, are plotted over time for the test systems according to Example 1 (▪, 1 cm²) and Example 2 (□, 1 cm²) in comparison to the reference system Transderm Scop (▴, 2.5 cm²) as mean value from six measurements ±standard deviation with use of dermatomed guinea pig skin.

In FIG. 2 the permeation profiles, determined as cumulative quantity of permeated scopolamine, are plotted over time for the test systems according to Example 3 (▪, 1 cm²) and Example 4 (□, 1 cm²) in comparison to the reference system Transderm Scop (▴, 2.5 cm²) as mean value from six measurements ±standard deviation with use of dermatomed guinea pig skin.

1. Transdermal therapeutic system without release-determining membrane for delivering scopolamine, comprising a skin contact layer, an active substance-containing reservoir layer, an active substance-impermeable backing layer, and optionally a protective layer which is detachable from the skin contact layer, wherein the skin contact layer is in direct contact with the active substance-containing reservoir layer.

2. Transdermal therapeutic system according to claim 1, characterised in that the active substance is present in the form of the free base of scopolamine.

3. Transdermal therapeutic system according to claim 1, characterised in that the active substance is present in dissolved form in the active substance-containing skin contact layer.

4. Transdermal therapeutic system according to claim 1, characterised in that the skin contact layer has an active substance content in the range of from approximately 1.2 to 13 wt. %.

5. Transdermal therapeutic system according to claim 1, characterised in that the reservoir layer has an active substance content in the range of from approximately 7 to 20 wt. %.

6. Transdermal therapeutic system according to claim 1, characterised in that it contains a total quantity of from approximately 0.8 to 2 mg.

7. Transdermal therapeutic system according to claim 1, characterised in that the skin contact layer and the reservoir layer are based on a self-adhesive, amine-resistant silicone adhesive polymer as base polymer.

8. Transdermal therapeutic system according to claim 1, characterised in that the reservoir layer and/or the skin contact layer contains permeation enhancers.

9. Transdermal therapeutic system according to claim 1, characterised in that the permeation enhancer is oleic acid, 2-(2-ethoxyethoxy)ethanol and/or is dipropylene glycol.

10. Transdermal therapeutic system according to claim 1, characterised in that the active substance is released from the transdermal therapeutic system over a period of time from 20 h to 72 h at a release rate of from 1.5 to 15 μg/cm²/h.

11. Transdermal therapeutic system according to claim 1, for use in the treatment of motion sickness and/or post-operative nausea.

12. Transdermal therapeutic system according to claim 1, characterised in that the reservoir layer and/or the skin contact layer contains a solubility enhancer and/or a thickening agent and/or a tack-improving additive.

13. Method for producing a scopolamine-containing transdermal therapeutic system according to claim 1, comprising the steps of:
a) applying, to an active substance-impermeable backing layer, a scopolamine-containing reservoir layer comprising scopolamine, an adhesive polymer and optionally permeation enhancers, wherein the scopolamine is either completely dissolved in the reservoir layer or is present in part in the form of non-dissolved particles,
b) applying, to a detachable protective layer, a scopolamine-containing skin contact layer comprising scopolamine, an adhesive polymer and optionally permeation enhancers as well as adhesive force-enhancing substances, wherein the scopolamine is present in dissolved form in the skin contact layer,
c) laminating the skin contact layer from b) onto the reservoir layer from a).

14. Transdermal therapeutic system according to claim 1, characterised in that the skin contact layer has an active substance content in the range of from approximately 9 to 11 wt. %.

15. Transdermal therapeutic system according to claim 1, characterised in that the reservoir layer has an active substance content in the range of from approximately 8 to 11 wt. %.

16. Transdermal therapeutic system according to claim 1, characterised in that it contains a total quantity of from approximately 1.2 to 1.5 mg scopolamine.

17. Transdermal therapeutic system according to claim 1, characterised in that the reservoir layer and/or the skin contact layer contains fatty acids, alcohols or ethers.

18. Transdermal therapeutic system according to claim 1, characterised in that the active substance is released from the transdermal therapeutic system over a period of time from 20 h to 72 h at a release rate of from 3 to 10 μg/cm²/h.

19. Transdermal therapeutic system according to claim 1, characterised in that the reservoir layer and/or the skin contact layer contains a silicone oil.
